(19) United States
(12) Patent Application Publication
(10) Pub. No.: US 2021/0154534 A1
(43) Pub. Date: May 27, 2021

(54) System And Method For Generating Graphical Visualization

(71) Applicant: Google LLC, Mountain View, CA (US)

(72) Inventors: Kent Eisenhuth, Wynnewood, PA (US); Ryan Kuykendall, Seattle, WA (US)

(21) Appl. No.: 16/695,405

(22) Filed: Nov. 26, 2019

(51) Int. Cl. A63B 43/00; A63B 37/00; A63B 45/02; A63B 24/00; A63B 43/06

(52) U.S. Cl. CPC A63B 43/008; A63B 37/0022; A63B 43/06; A63B 24/0021; A63B 37/0012; A63B 45/02

(57) ABSTRACT

A system is provided with one or more processors configured to receive inputs associated with a plurality of components of an organizational structure. For instance, the inputs may include an identifier, a first classification, and a second classification for each of the plurality of components. Based on the inputs, the one or more processors may determine relationships between the plurality of components within the organizational structure. The one or more processors may then generate a visualization with a plurality of graphic elements corresponding to the plurality of components, wherein the plurality of graphic elements are arranged based on the relationships between the plurality of components, and wherein the visualization includes one or more indications of the relationships between the plurality of components within the organizational structure.

### BACKGROUND

Complex architecture, such as organizational charts, technical architecture, process diagrams, or other systems with hierarchical structure, may be described by texts. For example, the hierarchical and cross linking relationships between features of an application, pages of a website, storages in a cloud computing system, devices in an electronic system, processes in a software program, topics in a book, departments of a corporation, etc., may be described by lengthy texts. To show the complex relationships, two-dimensional graphics, such as flowcharts, may be used to illustrate the connections and flow of information between the components within a complex architecture. In some instances, two-and-a-half dimensional (2.5D) graphics such as a two dimensional (2D) image that presents objects with simulated perspective or depth perception, or three dimensional (3D) rendered graphics, may be used as a visual aid for showing multi-dimensional relationships of components in a complex architecture. Further, animation may be used to provide better visualization of process or flow using a complex architecture.

### BRIEF SUMMARY

One aspect of the disclosure provides for receiving, by one or more processors, inputs associated with a plurality of components of an organizational structure, wherein the inputs include an identifier, a first classification, and a second classification for each of the plurality of components; determining, by the one or more processors based on the inputs, relationships between the plurality of components within the organizational structure; and generating, by the one or more processors, a visualization with a plurality of graphic elements corresponding to the plurality of components, wherein the plurality of graphic elements are arranged in a plurality of sections based on the first classifications, and arranged in one or more hierarchies based on the second classifications, and wherein the visualization includes one or more indications of the relationships between the plurality of components within the organizational structure.

The method may further comprise receiving, by the one or more processors, one or more additional classifications for each of the plurality of components; and generating, by the one or more processors, icons as the plurality of graphic elements, wherein appearance of the icons are based on the one or more additional classifications.

The method may further comprise receiving, by the one or more processors, additional input indicating that two or more of the plurality of components are connected; and generating, by the one or more processors based on the additional input, one or more connections for the corresponding graphic elements connecting to adjacent graphic elements in the visualization.

The method may further comprise receiving, by the one or more processors, additional input indicating that two or more of the plurality of components are crosslinked; and generating, by the one or more processors based on the additional input, one or more crosslinks for the corresponding graphic elements to non-adjacent graphic elements in the visualization. The one or more crosslinks may be hidden and appear in the visualization only when one or more of the crosslinked graphic elements is selected.

The method may further comprise receiving, by the one or more processors, one or more external links associated with one or more of the plurality of components, wherein the corresponding graphic elements are generated with the one or more external links.

The method may further comprise receiving, by the one or more processors, an address for each of the plurality of graphic elements, wherein the plurality of graphic elements are further arranged on the visualization based on the addresses.

The visualization may show the plurality of graphic elements as two-and-a-half or three dimensional objects in a perspective view.

The method may further comprise receiving, by the one or more processors, a command to change a view of the visualization; and generating, by the one or more processors based on the command, a two dimensional view of the visualization.

The inputs may be received in a tabular form.

The method may further comprise providing, by the one or more processors, a web-based application with templates for entering inputs, wherein the inputs associated with the plurality of components are received through the web-based application.

The method may further comprise receiving, by the one or more processors, an elevation for each of the plurality of components, wherein the plurality of graphic elements are further arranged on the visualization in a layered stack based on the elevations.

The method may further comprise receiving, by the one or more processors, additional inputs specifying one or more sequences including two or more of the plurality of components; and generating, by the one or more processors based on the additional inputs, one or more animated walkthroughs of graphic elements corresponding to the one or more sequences.

The one or more animated walkthroughs may be generated as an overlay on the visualization.

The inputs and the additional inputs may be received in a tabular form as separate sheets.

Another aspect of the disclosure provides for a system comprising one or more processors. The one or more processors are configured to receive inputs associated with a plurality of components of an organizational structure, wherein the inputs include an identifier, a first classification, and a second classification for each of the plurality of components; determine, based on the inputs, relationships between the plurality of components within the organizational structure; and generate a visualization with a plurality of graphic elements corresponding to the plurality of components, wherein the plurality of graphic elements are arranged based on the relationships between the plurality of components, and wherein the visualization includes one or more indications of the relationships between the plurality of components within the organizational structure.

The one or more processors may be further configured to receive one or more additional classifications for each of the plurality of components; and generate icons as the plurality of graphic elements, wherein appearance of the icons are based on the one or more additional classifications.

The one or more processors may be further configured to receive an elevation for each of the plurality of components, wherein the plurality of graphic elements are further arranged on the visualization in a layered stack based on the elevations.

The one or more processors may be further configured to receive additional inputs specifying one or more sequences including two or more of the plurality of components; and generate, based on the additional inputs, one or more animated walkthroughs of graphic elements corresponding to the one or more sequences.

Still another aspect of the disclosure provides for a computer-readable storage medium storing instructions executable by one or more processors for performing a method. The method comprises receiving inputs associated with a plurality of components of an organizational structure, wherein the inputs include an identifier, a first classification, and a second classification for each of the plurality of components; determining, based on the inputs, relationships between the plurality of components within the organizational structure; and generating a visualization with a plurality of graphic elements corresponding to the plurality of components, wherein the plurality of graphic elements are arranged in a plurality of sections based on the first classifications, and arranged in one or more hierarchies based on the second classifications, wherein the visualization includes one or more indications of the relationships between the plurality of components within the organizational structure.

### BRIEF DESCRIPTION OF THE DRAWINGS

FIG. 1 is a block diagram showing an example system for generating visualization in accordance with aspects of the disclosure.

FIG. 2 is an example flow diagram for generating visualization in accordance with aspects of the disclosure.

FIG. 3 shows example inputs in a tabular form in accordance with aspects of the disclosure.

FIG. 4 shows an example visualization generated using the inputs of FIG. 3 in accordance with aspects of the disclosure.

FIG. 5 shows an example 2D view of the visualization 400 of FIG. 4 in accordance with aspects of the disclosure.

FIG. 6 shows example inputs in a tabular form in accordance with aspects of the disclosure.

FIG. 7 shows an example visualization generated using the inputs of FIG. 6 in accordance with aspects of the disclosure.

FIG. 8 shows another example visualization generated using the inputs of FIG. 6 in accordance with aspects of the disclosure.

FIG. 9 shows another example visualization generated using the inputs of FIG. 6 in accordance with aspects of the disclosure.

FIG. 10 shows example inputs in a tabular form in accordance with aspects of the disclosure.

FIG. 11 shows an example overview of an animated visualization generated using the inputs of FIG. 10 in accordance with aspects of the disclosure.

FIGS. 12A-12C show example snapshots in the animated visualization generated using the inputs of FIG. 10 in accordance with aspects of the disclosure.

FIG. 13 shows another example overview of another example animated visualization in accordance with aspects of the disclosure.

### DETAILED DESCRIPTION

### Overview

The technology generally relates to a visualization tool. A complex architecture may not be easily described using textual information alone. A flowchart may be helpful in illustrating relationships and/or flow of information, but may require many branches or even multiple flowcharts for a complex architecture. While 2.5 or 3 dimensional graphics and animations may provide better visualization of a complex architecture, they may require significant design skills and/or effort to generate. To address these issues, a system including one or more processors may be configured to generate visualizations based on simple inputs.

In this regard, a system including one or more processors may be configured to receive inputs associated with a plurality of components of an organizational structure. For instance, the plurality of components may be part of any organizational structure including one or more hierarchies. For example, the plurality of components may be features of an application, pages of a website, storages in a cloud computing system, devices in an electronic system, processes in a software program, topics in a book, departments in a corporation, locations in a tour, etc. The inputs may be provided in a simple form, such as in a tabular form. The inputs may include an identifier, a first classification, and a second classification for each of the plurality of components. For example, the identifier may be a name of each feature, webpage, storage, device, process, topic, departments, locations, etc. The first classification may be a category of the feature, webpage, storage, device, process, topic, department, location, etc., and the second classification may be a hierarchy level of the feature, webpage, storage, device, process, topic, department, location, etc. within the respective category.

Based on the inputs, the system may determine relationships between the plurality of components within the organizational structure. For example, the system may determine which of the features, pages, storages, devices, processes, topics, departments, locations, etc. belong in the same categories, and how they relate to one another in one or more hierarchies.

The system may then generate a visualization with a plurality of graphic elements corresponding to the plurality of components, wherein the plurality of graphic elements are arranged in a plurality of sections based on the first classifications, and arranged in one or more hierarchies based on the second classifications. For example, the system may then generate icons corresponding to the various features, webpages, storages, devices, processes, topics, departments, locations, etc., and arrange the icons relative to one another based on the relationships between the various features, pages, storages, devices, processes, topics, departments, locations, etc. The visualization may include one or more indications of the relationships between the plurality of components within the organizational structure.

In some instances, the system may be further configured to receive additional inputs specifying one or more sequences including two or more of the plurality of components. For example, the additional inputs may be received in a tabular form. Based on the additional inputs, the system may generate one or more animated walkthroughs of graphic elements corresponding to the one or more sequences. For example, the walkthrough may be an example interaction of a user with features of an application or pages of a website, an example flow of information through storages for a cloud computing system, an example operation using a sequence of devices, an example run of processes in a software program, an example storyline, a product being developed through various departments of a corporation, an example tour itinerary, etc.

The technology is advantageous because it provides visualization of complex architecture in 2.5D or 3D views. Features of the technology allow users to provide simple inputs, based on which complex illustrative visualizations may be generated quickly, instead of requiring significant design skills and/or effort. The technology further allows users to generate animations of user experience and/or flow of information using simple inputs, also without requiring significant design skills and/or effort.

### Example Systems

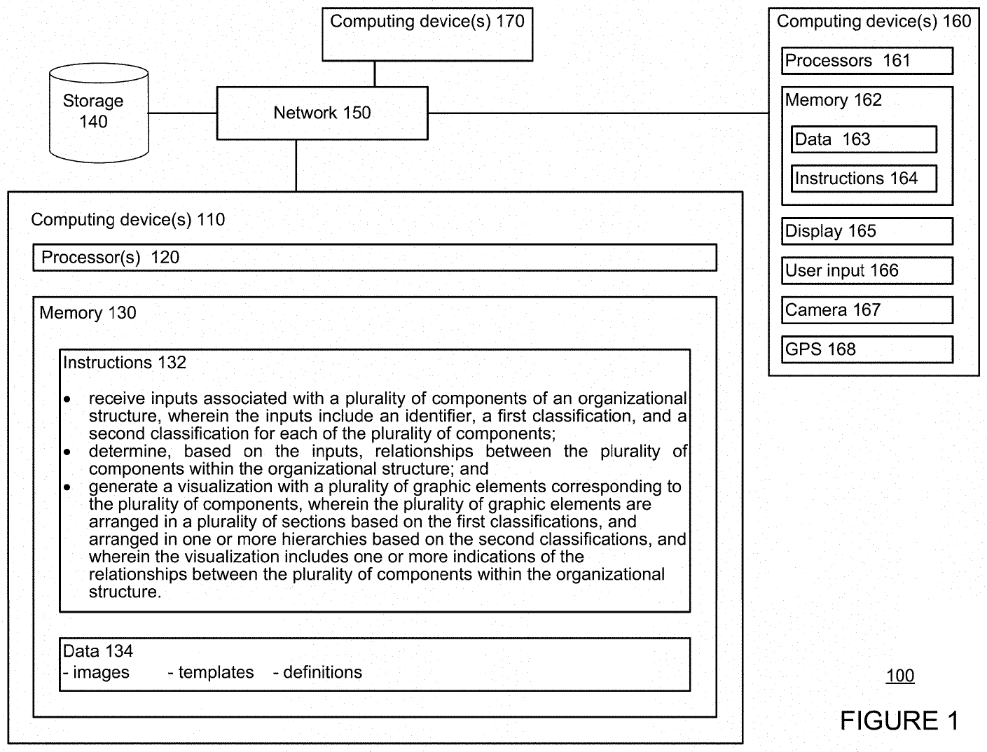

FIG. 1 illustrates an example system used to generate a visualization with graphic components. It should not be considered as limiting the scope of the disclosure or usefulness of the features described herein. In this example, system 100 can include one or more computing devices 110 in communication with one or more computing devices 160, 170, as well as storage system 140, through a network 150. Each of the computing devices 110, 160, 170 may contain one or more processors, memory, and other components typically present in general purpose computing devices.

For instance, the one or more processors 120 can be any conventional processors, such as a commercially available CPU. Alternatively, the processors can be dedicated components such as an application specific integrated circuit (“ASIC”) or other hardware-based processor. Although not necessary, the computing devices 110 may include specialized hardware components to perform specific computing processes.

The memory 130 of the computing devices 110 can store information accessible by the one or more processors 120, including instructions 132 and data 134. The memory can be of any non-transitory type capable of storing information accessible by the processor, such as a hard-drive, memory card, ROM, RAM, DVD, CD-ROM, write-capable, and read-only memories.

The instructions 132 can be any set of instructions to be executed directly, such as machine code, or indirectly, such as scripts, by the one or more processors 120. In that regard, the terms “instructions,” “application,” “steps,” and “programs” can be used interchangeably herein. The instructions can be stored in object code format for direct processing by a processor, or in any other computing device language including scripts or collections of independent source code modules that are interpreted on demand or compiled in advance. Functions, methods, and routines of the instructions are explained in more detail below. For instance, the instructions 132 may include how to process data, generate visualizations, generate animations, etc.

The data 134 may be retrieved, stored, or modified by the one or more processors 120 in accordance with the instructions 132. For instance, although the subject matter described herein is not limited by any particular data structure, the data can be stored in computer registers, in a relational database as a table having many different fields and records, or XML documents. The data can also be formatted in any computing device-readable format such as, but not limited to, binary values, ASCII or Unicode. Moreover, the data can comprise any information sufficient to identify the relevant information, such as numbers, descriptive text, propriety codes, pointers, references to data stored in other memories such as at other network locations, or information that is used by a function to calculate the relevant data. For instance, the data 134 may include images, definitions, templates, etc.

Although FIG. 1 functionally illustrates the processor, memory, and other elements of computing devices 110 as being within the same block, the processor, computer, computing device, or memory can actually comprise multiple processors, computers, computing devices, or memories that may or may not be stored within the same physical housing. For example, the memory can be a hard drive or other storage media located in housings different from that of the computing devices 110. Accordingly, references to a processor, computer, computing device, or memory will be understood to include references to a collection of processors, computers, computing devices, or memories that may or may not operate in parallel. For example, the computing devices 110 may include server computing devices operating as a load-balanced server farm, distributed system, etc. Yet further, although some functions described below are indicated as taking place on a single computing device having a single processor, various aspects of the subject matter described herein can be implemented by a plurality of computing devices, for example, communicating information over network 150.

Each of the computing devices 110, 160, 170 can be at different nodes of a network 150 and capable of directly and indirectly communicating with other nodes of network 150. Although only a few computing devices are depicted in FIG. 1, it should be appreciated that a typical system can include a large number of connected computing devices, with each different computing device being at a different node of the network 150. The network 150 and intervening nodes described herein can be interconnected using various protocols and systems, such that the network can be part of the Internet, World Wide Web, specific intranets, wide area networks, or local networks. The network can utilize standard communication protocols, such as Ethernet, WiFi and HTTP, protocols that are proprietary to one or more companies, and various combinations of the foregoing. Although certain advantages are obtained when information is transmitted or received as noted above, other aspects of the subject matter described herein are not limited to any particular manner of transmission of information.

As an example, computing devices 110 may be server computing devices, while computing devices 160, 170 may be client computing devices. For instance, computing devices 110 may include web servers capable of communicating with storage system 140 as well as computing devices 160, 170 via the network 150. For example, computing devices 110 may be server computing devices that can use network 150 to transmit and present information to a user on a display, such as display 165 of computing device 160.

Each of the client computing devices 160, 170 may be configured similarly to the server computing devices 110, with one or more processors, memory and instructions as described above. Each of the client computing devices 160, 170 may be a personal computing device intended for use by a user, and have all of the components normally used in connection with a personal computing device. For example as shown, client computing device 160 includes processors 161 (e.g., a central processing unit CPU), memory 162 (e.g., RAM and internal hard drives) storing data 163 and instructions 164, a display such as display 165 (e.g., a monitor having a screen, a touch-screen, a projector, a television, or other device that is operable to display information), and user input device 166 (e.g., a mouse, keyboard, touch-screen, or microphone). The client computing device 160 may also include a camera 167 for recording video streams and/or capturing images, speakers, a network interface device, and all of the components used for connecting these elements to one another. The client computing device 160 may also include a location determination system, such as a GPS 168. Other examples of location determination systems may determine location based on wireless access signal strength, images of geographic objects such as landmarks, semantic indicators such as light or noise level, etc.

Although the client computing devices 160, 170 may each comprise a full-sized personal computing device, they may alternatively comprise mobile computing devices capable of wirelessly exchanging data with a server over a network such as the Internet. By way of example only, client computing device 160, 170 may be a mobile phone or a device such as a wireless-enabled PDA, a tablet PC, a netbook, a smart watch, a head-mounted computing system, or any other device that is capable of obtaining information via the Internet. As an example the user may input information using a small keyboard, a keypad, microphone, using visual signals with a camera, or a touch screen.

As with memory 130, storage system 140 can be of any type of computerized storage capable of storing information accessible by the server computing devices 110, such as a hard-drive, memory card, ROM, RAM, DVD, CD-ROM, write-capable, and read-only memories. In addition, storage system 140 may include a distributed storage system where data is stored on a plurality of different storage devices which may be physically located at the same or different geographic locations. As shown, storage system 140 may be connected to various computing devices via the network 150, and/or may be directly connected to any of the computing devices 110, 160, 170.

### Example Methods

Further to example systems described above, example methods are now described. Such methods may be performed using the systems described above, modifications thereof, or any of a variety of systems having different configurations. It should be understood that the operations involved in the following methods need not be performed in the precise order described. Rather, various operations may be handled in a different order or simultaneously, and operations may be added or omitted.

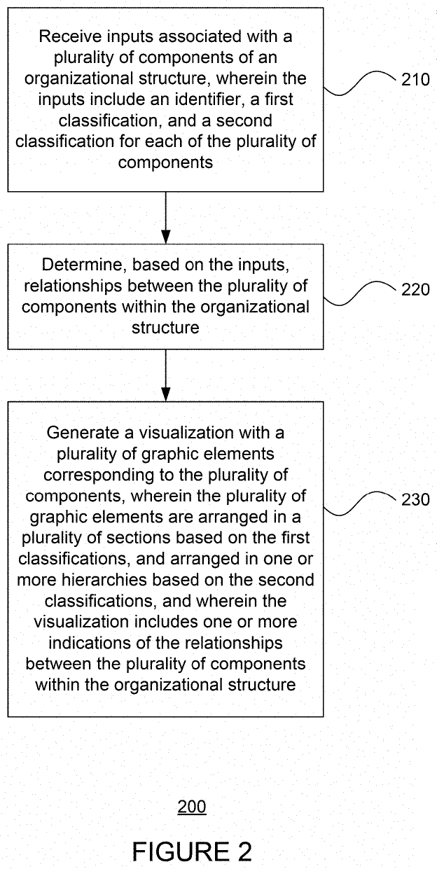

FIG. 2 shows an example flow diagram 200 for generating a visualization with graphic components. Flow diagram 200 may be performed by one or more processors, such as one or more processors 120 shown in FIG. 1. For example, processors 120 may receive data and make various determinations as shown in the flow diagram 200.

Referring to FIG. 2, at block 210, inputs associated with a plurality of components of an organizational structure are received, wherein the inputs include an identifier, a first classification, and a second classification for each of the plurality of components. The inputs may be received by processors 120. For example, inputs may be provided via user input 166 of computing device 160, and processors 161 may provide the inputs to processors 120. In some instances, processors 120 may generate a web-based application, which may provide templates for entering inputs. The templates may be viewed on display 165 and downloaded to computing device 160. The templates may be modified with inputs via user input 166. The modified templates may be uploaded to the web-based application, or an address (e.g. URL) of the modified template may be provided to the web-based application. Processors 120 may then access the inputs in the modified template.

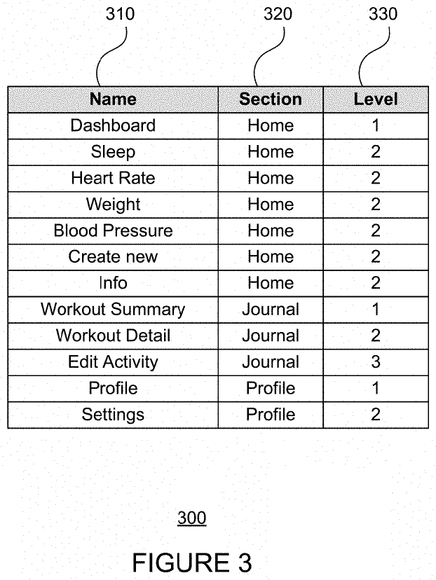

The inputs may be received by processors 120 in a simple form, such as in a tabular form or some other textual form. FIG. 3 illustrates example inputs in a tabular form. The example shown is for an exercise tracking application, where the table 300 includes inputs associated with a plurality of components of the exercise tracking application. As shown, the plurality of components of the exercise tracking application includes components that are displayed on multiple graphic user interfaces (GUIs). Although a particular example is shown, the plurality of components may be part of any organizational structure including one or more hierarchies. The plurality of components may generally be components in an organizational chart, technical architecture, process diagram, or any hierarchical structure. For example, the plurality of components may be pages of a website, storages for a cloud computing system, parts in an electronic device, processes in a software, topics of a book, departments of a corporation, physical or virtual locations, etc.

As shown in table 300, inputs on the plurality of components may include identifiers 310. For example, the identifiers 310 may be provided in a “Name” column of the table 300. Thus, names of each feature of the exercise tracking application, such as “Dashboard,” “Heart Rate,” “Info,” “Settings,” etc., may be provided as identifiers 310. In other examples, the identifiers may be names of webpages, storages, devices, processes, topics, departments, locations, etc.

Table 300 further shows that the inputs may include one or more classifications for each of the plurality of components, such as first classification 320 and second classification 330. The first and second classifications 320, 330 may define relationships between the various components. As shown, the first classification 320 may be provided in a “Section” column, which categorizes the components. For instance, “Dashboard,” “Sleep,” “Heart Rate” may all be features shown on one GUI of the exercise tracking application, and are given the first classification “Home,” “Workout Summary,” “Workout detail,” and “Edit Activity” may be features shown on another GUI of the exercise tracking application, and given the first classification of “Journal,” and “Profile” and “Settings” may be features shown on yet another GUI of the exercise tracking application, and given the first classification of “Profile.”

Further as shown, the second classification 330 may be provided in a “Level” column, which specifies a hierarchy of components within a category. For example, within the “Journal” GUI, “Workout Summary” may be on level 1, which may be an initial screen that a user sees in the “Journal” GUI, then “Workout Detail” is on level 2, which may be a second screen that the user sees in the “Journal” GUI, for example a display of “Workout Detail” may be opened from a display of “Workout Summary,” and “Edit Activity” is on level 3, which may be a third screen that the user sees in the “Journal” GUI. In other examples, the first classification 320 may be categories of webpages, storages, devices, processes, departments, topics, locations, etc., and the second classification 330 may specify the hierarchies of the webpages, storages, devices, processors, departments, topics, locations, etc. within each category.

Referring back to FIG. 2, at block 220, relationships between the plurality of components within the organizational structure are determined based on the inputs. For instance, based on the first classification, the plurality of components may be grouped into categories, and based on the second classification, hierarchy of components within a category may be determined. Thus in the example of FIG. 3, based on the inputs in table 300, processors 120 may determine that the example exercise tracking application include features in three different categories, each has its own hierarchy with multiple levels. For instance, processors 120 may determine that the “Home” GUI includes 7 displays in 2 levels, the “Journal” GUI includes 3 displays in 3 levels, and the “Profile” GUI includes 2 displays in 2 levels. In other examples, the processors 120 may determine hierarchical relationship between webpages, storages, devices, processes, departments, topics, locations, etc. within each respective category.

Referring again to FIG. 2, at block 230, a visualization is generated with a plurality of graphic elements corresponding to the plurality of components. The plurality of graphic elements are arranged in a plurality of sections based on the first classifications, and arranged in one or more hierarchies based on the second classifications. The visualization may include one or more indications of the relationships between the plurality of components within the organizational structure. For instance, the processors 120 may generate a graphic element corresponding to each of the plurality of components in the received inputs. The processors 120 may generate a visualization with the graphic elements arranged based on the relationships determined using the first and second classifications, such as arranging in different sections and hierarchies, which may include indications of relationships between the plurality of components. The processors 120 may then provide the visualization for display, for example on display 165 of computing device 160. Alternatively or additionally, the processors 120 may provide the visualization in a web-based application or at an address (e.g. URL) accessible by computing device 160, or export the visualization as one or more images.

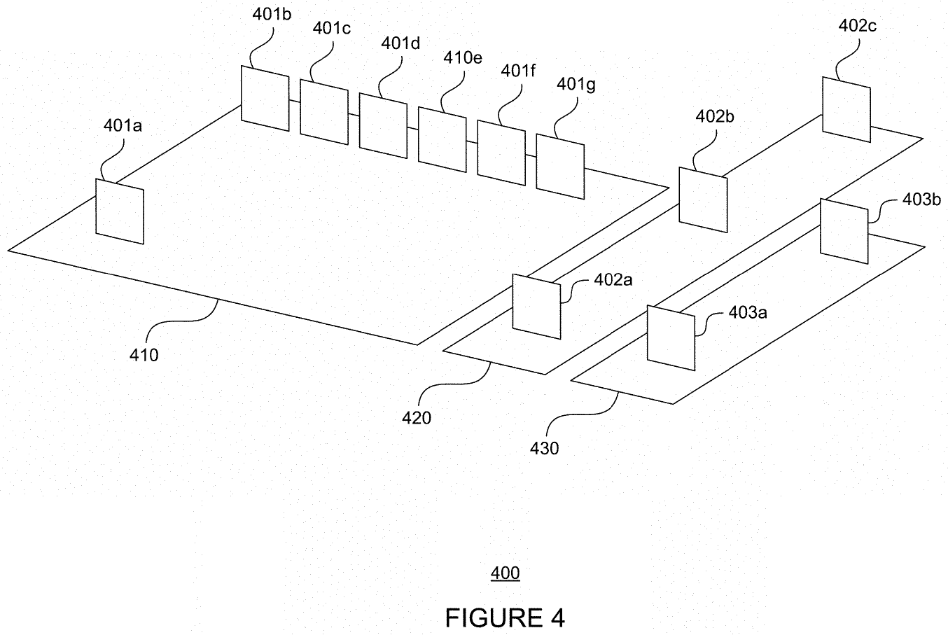

FIG. 4 shows an example visualization 400 generated based on the example inputs shown in FIG. 3. As shown, a plurality of graphic elements 401a-401g, 402a-402c, and 403a-403b, are generated corresponding to the plurality components listed in table 300. In this example, the graphic elements are icons. Although the icons are shown in this example are rectangular in shape, in other examples the icons may be any other shape, such as circle, oval, polygon, arbitrary shape, etc. The graphic elements 401a-401g may each further include the respective identifiers 310 or otherwise identify the corresponding component. For example, though not shown, the graphic element 401a may further include the text “Dashboard” on or near the icon, and/or may include an image of the Dashboard in the icon.

The graphic elements 401a-403c are shown arranged based on the first and second classifications 320, 330 in the table 300. For example, based on the first classification 320, graphic elements 401a-401g corresponding to components “Dashboard,” “Sleep,” “Heart Rate,” “Weight,” “Blood Pressure,” “Create New,” and “Info” may be arranged in a first section 410 corresponding to the category “Home”; graphic elements 402a-402c corresponding to components “Workout Summary,” “Workout Detail,” and “Edit Activity” may be arranged in a second section 420 corresponding to the category “Journal”; and graphic elements 403a-403b corresponding to components “Profile” and “Settings” may be arranged in a third section 430 corresponding to the category “Profile.” The sections may be shown distinguished by the separate regions they occupy in the visualization 400. Alternatively or additionally, the sections may be distinguished in any other way, such as having different colors, shading, format, etc.

Further as shown, based on the second classification 330, graphic elements 401a-401g within the first section 410 are arranged based on their hierarchical relationship within the category “Home,” graphic elements 402a-402c within the second section 420 are arranged based on their hierarchical relationship within the category “Journal,” and graphic elements 403a-403b within the third section 430 are arranged based on their hierarchical relationship within the category “Profile.” In this regard, the hierarchy levels may be represented by the positioning of the graphic elements within each respective section. In the example visualization 400, graphic elements in a same hierarchy level are arranged in a same row. Alternatively or additionally, the hierarchy levels may be distinguished in any other way, such as arranged in different columns, having different colors, shading, format, etc. Further as shown, the relationships between the plurality of components of FIG. 3 are indicated by the relative positions of the plurality of graphic elements 401a-401g, 402a-402b, and 403a-403b.

Once generated, the visualization 400 may be provided for display. FIG. 4 shows the visualization 400 in a 2.5D view. That is, the 2.5D view presents the graphic elements 401a-403b in a two dimensional image, but with a simulated appearance of perspective or depth perception. Alternatively, the graphic elements 401a-403b may be rendered as three dimensional objects arranged in a three dimensional space and viewed from a perspective view. In some instances, the display may be interactive, such as allowing for zooming in and/or out, changing of perspective, etc. Alternatively or additionally, when a command is received, a two dimensional view of the visualization 400 may be generated and displayed. The processors 120 may generate the 2D view based on the inputs of FIG. 3, for example generating branches in a tree or nodes on a map based on the first and second classifications 320 and 330. Alternatively, the processors 120 may generate the 2D view based on the visualization 400, such as rotating visualization 400 to a birds-eye view.

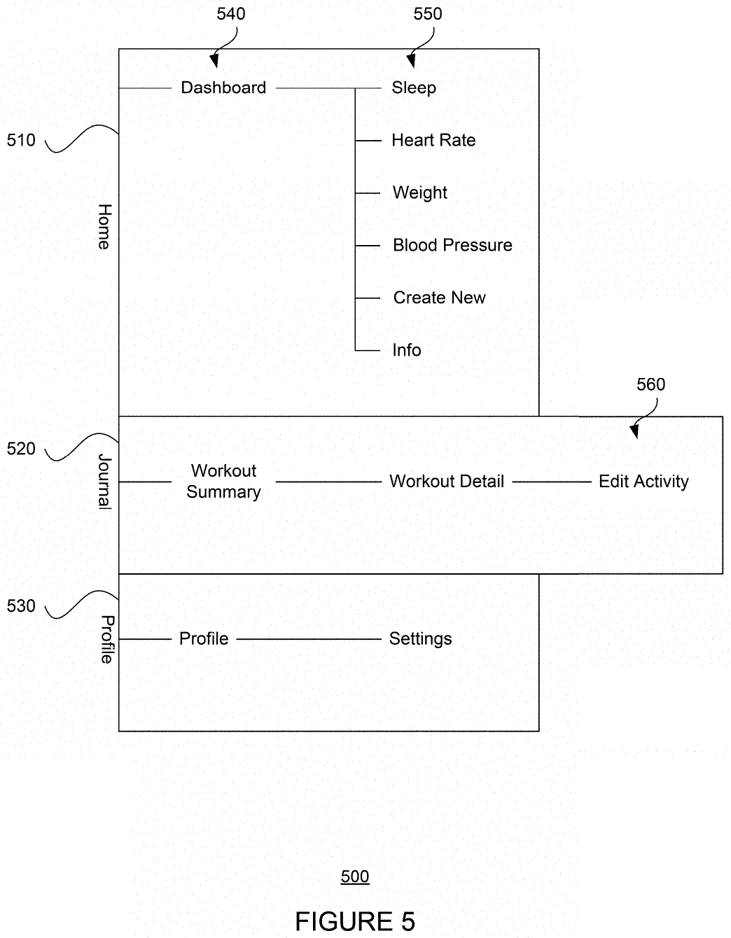

FIG. 5 shows an example 2D view 500 generated for the visualization 400. The 2D view in this example is 2D chart. In other examples, the 2D view may be in any other format, such as a tree format, a map format, a flowchart format, etc. Texts corresponding to the identifiers 310 are shown in the 2D view, organized based on the first classification 320 into three sections 510, 520, 530, and further organized based on the second classification 330 into three hierarchy levels 540, 550, 560. The 2D view may optionally include other features, such as additional texts, icons, images, designs, etc. Thus, based on a same set of inputs in table 300, different forms of visualizations may be generated by processors 120, including the 2.5D visualization 400 of FIG. 4 and the 2D view 500 of FIG. 5.

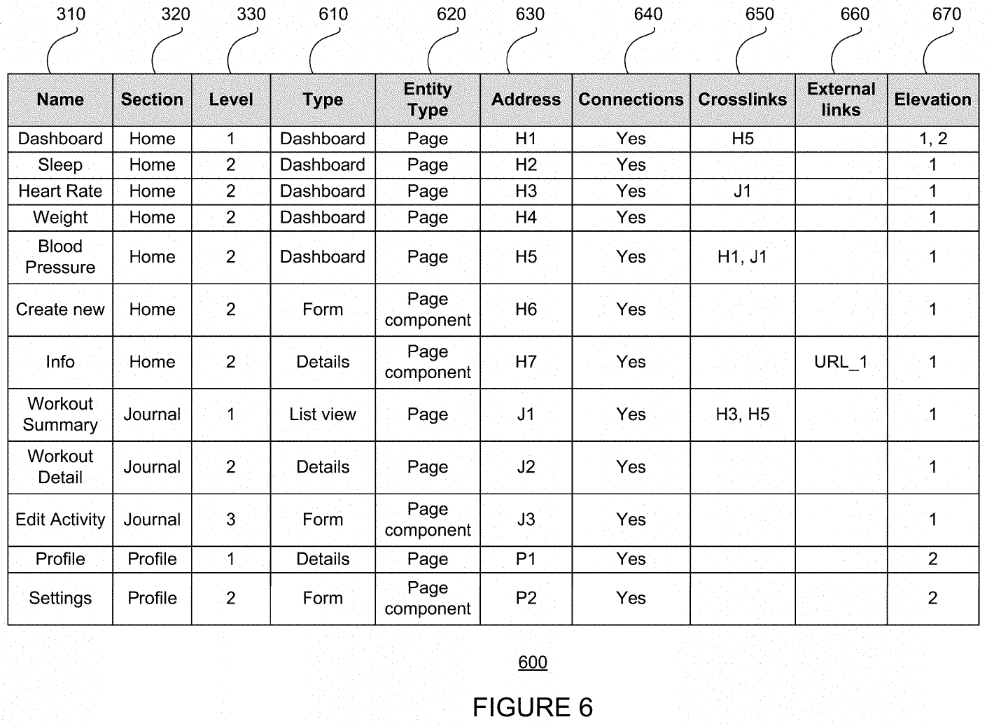

In some instance, the inputs received by processors 120 may include other information in addition to the identifiers and first and second classifications, based on which a visualization may be generated. FIG. 6 shows a table 600 with many similar features as table 300 of FIG. 3, and are labeled as such. However, table 600 includes additional inputs not included in table 300 of FIG. 3. In this regard, where processors 120 provide multiple templates for entering inputs, some or each of the templates may allow different types of additional information to be provided as input. Alternatively or additionally, templates may be customizable and allow new categories of additional information to be created.

Referring to FIG. 6, the inputs in table 600 may include one or more additional classifications, such as a third classification 610 and a fourth classification 620. For instance, the third classification 610 may be provided in a “Type” column, which specifies whether each component is of a type “Dashboard,” “Form,” “Details,” or “List View.” The fourth classification 620 may be provided in an “Entity Type” column, which specifies whether a component is a page or a component of a page. Though not shown, the third and fourth classifications 610 and 620 may include alternative and/or additional choices. For example, the third classification 610 may further include types such as “Graph,” “Folder,” “Texts,” “Image,” etc., and/or the fourth classification 620 may include whether a component is multipage or single page, a pop-out window, etc. Still further, any other alternative or additional classifications may be provided as inputs. In this regard, the classifications available may depend on the type of architecture to be visualized, and may be provided as templates. For example, the additional classifications may further classify types of webpages, storages, devices, processes, departments, topics, locations, etc.

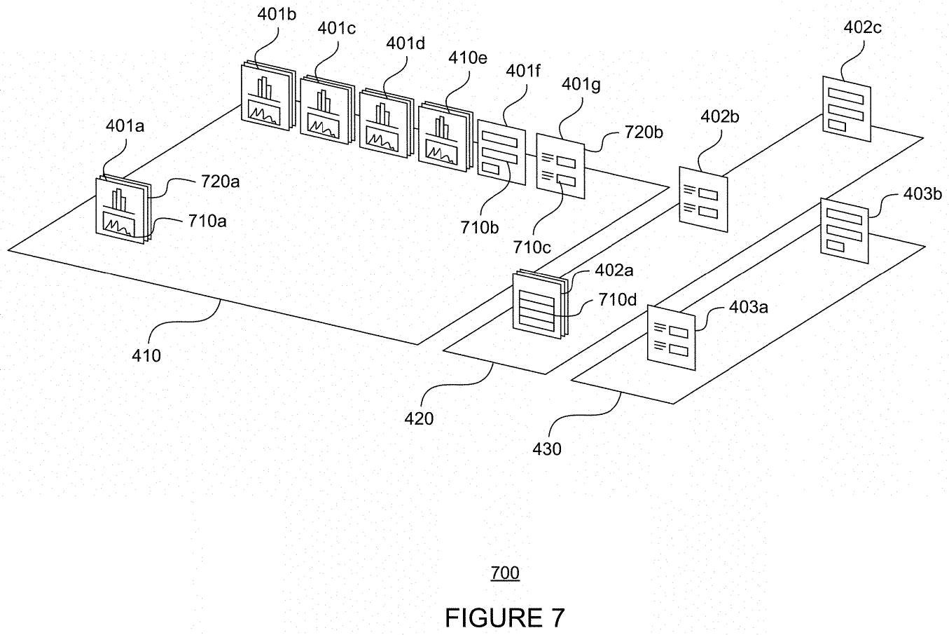

FIG. 7 shows an example visualization 700 generated based on the example inputs shown in FIG. 6. Visualization 700 includes many similar features as visualization 400, and are labeled as such. However, visualization 700 is generated further based on the third and fourth classifications 610 and 620 in table 600. In particular, the graphic elements 401a-401g, 402a-402c, and 403a-403b are generated with appearances based on the third and fourth classifications 610 and 620.

Thus as shown, since the components “Dashboard,” “Sleep,” “Heart Rate,” “Weight” and “Blood Pressure” each have a third classification of “Dashboard,” graphic elements 401a-401e corresponding to these components each have an appearance 710a corresponding to this third classification, shown as a chart and a graph. Since the components “Create New,” “Edit Activity,” and “Settings” each have a third classification of “Form,” graphic elements 401f, 402c, and 403b corresponding to these components each have an appearance 710b corresponding to this third classification, shown as a form with fillable fields. The components “Info,” “Workout Detail,” and “Profile” each have a third classification of “Details,” thus the graphic elements 401g, 402b, and 403a corresponding to these components each have an appearance 710c corresponding to this third classification, shown as some texts and other items. The component “Workout Summary” has a third classification of “List View,” thus the graphic element 402a corresponding to this component has an appearance 710d corresponding to this third classification, shown as a list of items.

Further as shown, since the components “Dashboard,” “Sleep,” “Heart Rate,” “Weight,” “Blood Pressure,” and “Workout Summary” each have a fourth classification of “Page,” elements 401a-401e and 402a corresponding to these components each have an appearance 720a corresponding to this fourth classification, shown as multiple icons. In contrast, since the components “Create New,” “Info,” “Workout Detail,” “Edit Activity,” “Profile,” and “Settings” each have a fourth classification of “Page component,” graphic elements 401f-401g, 402b-402c, 403a-403b corresponding to these components each have an appearance 720b corresponding to this fourth classification, shown as a single icon. As alternative or in addition to the example appearances shown in FIG. 7, the graphic elements may have other appearances, which may include use of different colors, shading, shapes, texts, images, symbols, etc. In some instances, inputs in table 600 may specify brand-specific format or styling of the graphic elements in the visualization. In some instances, inputs in table 600 may specify brand-specific format or styling for the graphic elements, based on which visualization with brand-specific format or styling may be generated.

Returning to FIG. 6, the inputs in table 600 may further include additional information on the relationships of the plurality of components with each other and with external components, which may be specified by address 630, connections 640, crosslinks 650, and external links 660. For instance, relative positioning of graphic elements representing the plurality of components may be provided in the “Address” column of table 600. The address 630 may have any naming convention. In the example shown, the addresses include a first letter of the section, and a number indicating an order of the component within the section. Thus address “H1” is provided for “Dashboard,” which is the first component in the section “Home.” Other naming conventions may be used, which for example may include only numbers or letters, or include punctuation, or may also specify the hierarchy level.

Further as shown, table 600 may also include information on how the graphic elements should be connected or linked. For instance, whether graphic elements representing the plurality of components should be connected in the visualization may be provided in the “Connections” column of table 600. Inputs for the connections 640 may be provided as a binary yes/no indicating whether connections should exist between adjacent graphic elements as shown in FIG. 6, or alternatively may specify the particular connections to be made (not shown). Further, whether graphic elements that are not adjacent should be crosslinked may be provided in the “Crosslinks” column of table 600. Inputs for the crosslinks 650 may be provided using address of the component to be crosslinked as shown, or alternatively may be provided using the identifier of the component to be crosslinked (not shown). Still further, whether any component is associated with an external component may be provided in the “External links” column of table 600. Inputs for the external links 660 may be provided using an external address, such as an URL, of the associated external component.

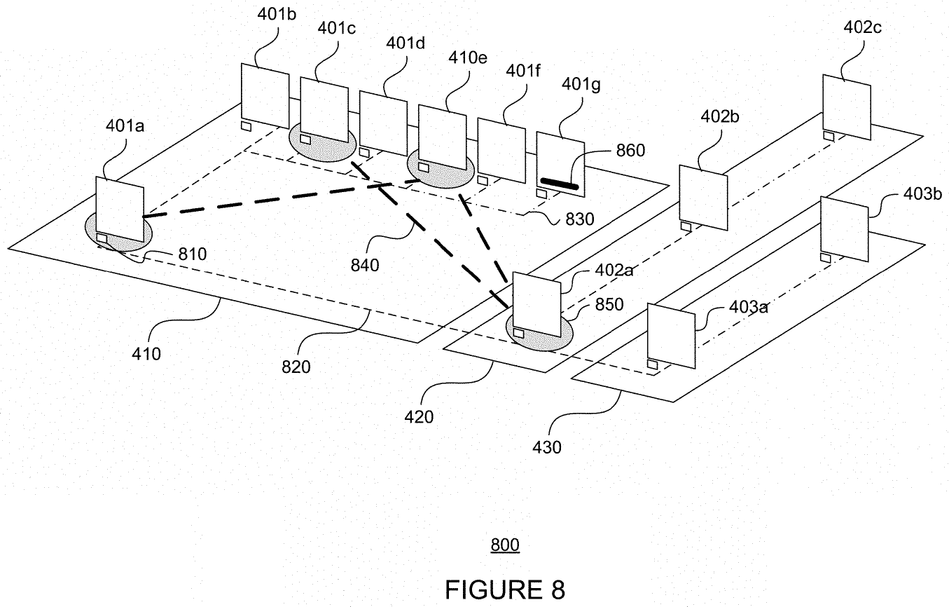

FIG. 8 shows another example visualization 800 generated based on the example inputs shown in FIG. 6. Visualization 800 includes many similar features as visualization 400, and are labeled as such. However, visualization 800 is generated further based on the relationship information in table 600. In particular, the visualization 800 further shows relationships between the graphic elements 401a-401g, 402a-402c, and 403a-403b, as well as to external components.

As shown in FIG. 8, an address 810 is shown positioned near each graphic element 401a-401g, 402a-402c, and 403a-403b based on the address 630 in table 600. Although the address 810 is shown below each icon, in other examples the address may be positioned above the icon, on either side of the icon, or on the icon itself.

FIG. 8 further shows that graphic elements are connected based on the inputs in connections 640 in table 600. For example, graphic elements within a same section, such as graphic elements 401a-401g may be connected, graphic elements 402a-402c may be connected, and graphic elements 403a-403b may be connected. Optionally as shown, the graphic elements 401a, 402a, and 403a corresponding to components in the lowest hierarchy may be connected. In some instances as shown, the connections may have different formats based on one or more classifications of the plurality of components, such as the first, second, third or fourth classification 320, 330, 610, 620. For example, connections 820 between components having the fourth classification of “page” may have one format (shown as dash lines), while connections 830 between components having the fourth classification of “page component” may have another format (shown as dot-dash lines).

Crosslinks 840 between graphic elements that are not adjacent in the visualization may be generated in FIG. 8 based on the inputs in crosslinks 650 in table 600. As shown, crosslinks may be between graphic elements within a same section that are not adjacent, such as graphic elements 401a and 401e, or between graphic elements in different sections, such as between graphic element 402a and graphic elements 401c and 401e. In some instances such as shown in FIG. 8, the crosslinks 840 may have a different format (shown as bold dash lines) as the connections between graphic elements in the same section. Optionally, the crosslinks 840 may appear only when one of the crosslinked graphic elements is selected. For example, selection may be when a mouse cursor is in an area of the graphic element or when the graphic element is clicked. Still further, when a crosslinked graphic element is selected, highlights 850 may be provided for the selected graphic element and the crosslinked graphic elements.

Further as shown in FIG. 8, an external link 860 is shown positioned on graphic element 401g based on inputs in external links 660 in table 600. Although the external link 860 is shown as a hyperlink on the icon, in other examples the hyperlink may be positioned above, below or on either side of the icon. In other examples, instead of showing the external link 860, graphic element 401g corresponding to an externally linked component may be provided in a different format, such as having a different color, shape, shading, image, symbol, etc.

Again returning to FIG. 6, the inputs in table 600 may still further include additional information for generating a layered stack visualization. For instance, relative elevation of graphic elements representing the plurality of components may be provided in the “Elevation” column of table 600. The elevation 670 may have any convention. In the example shown, the elevation 670 is indicated by a number, where a smaller number indicates a lower elevation. Thus in this particular example, some components with the first classification of “Home” and all components with the first classification of “Profile” are given an elevation of 1, while one component with the first classification of “Home” and all components with the first classification of “Profile” are given an elevation of 2. Thus as shown, components having the same one or more classifications need not be given a same elevation, as such, the elevations may present yet another dimension of the relationships between the plurality of components. Further, some components may be given more than one elevation. Other conventions for specifying elevation may be used, which for example may include only number, letters, punctuations, or any combination thereof.

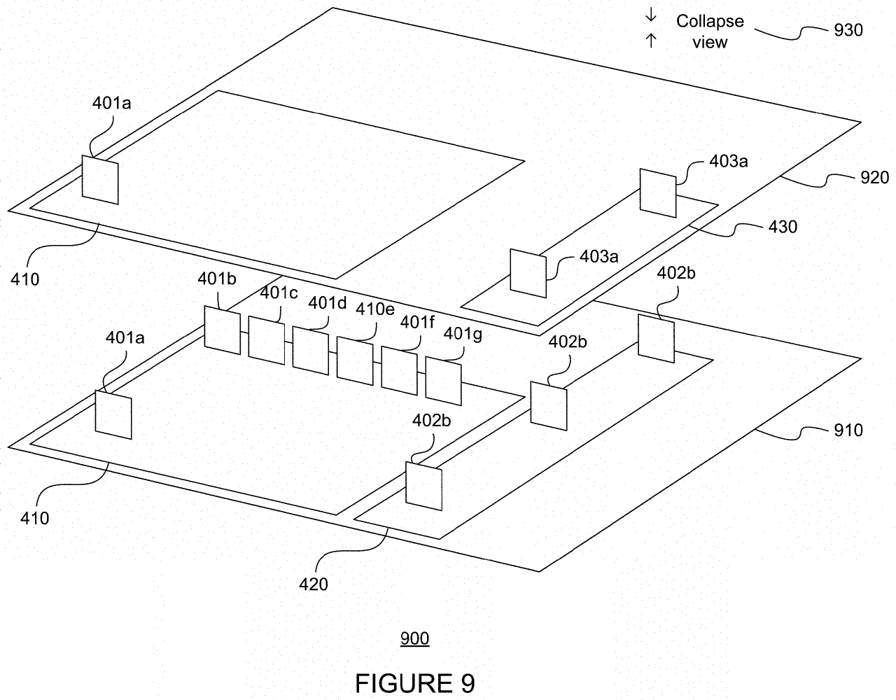

FIG. 9 shows another example visualization 900. Visualization 900 includes many similar features as visualization 400, and are labeled as such. However, visualization 900 is generated further based on the elevation information in table 600. In particular, the visualization 900 further illustrate relationships between the graphic elements 401a-401g, 402a-402c, and 403a-403b by arranging them in multiple elevation levels. Although two elevation levels are shown in this example, in other examples the visualization may include any number of elevation levels.

As shown, the graphic elements 401a-401g, 402a-402c, and 403a-403b are arranged in two elevation levels 910 and 920. Graphic elements in the first section 410 and in the second section 420 are shown arranged in the first elevation level 910. The second elevation level 920 includes graphic elements 403a-403b and only one graphic element 401a from the first section 410. In this regard, each elevation level may highlight one or more aspects of the architecture. For example, graphic elements arranged in the second elevation 920 may illustrate the screens that a user navigates through to make administrative changes. The visualization 900 further provides collapse view option 930 to change the view back to a single elevation visualization. Alternatively or additionally, the multi-level visualization 900 may further illustrate another dimension of relationships between the plurality of components. For example, replicas in a cloud computing system may be shown in multiple elevation levels. For another instance, parts in an electronic device performing different functions may be shown in different elevation levels, and pathways connecting the different functions may be shown between the elevations levels.

In another aspect, one or more animations may be generated for the visualization. In this regard, additional inputs may be received including one or more sequences of components to be animated in walkthroughs. The inputs on the animated walkthroughs may be received by processors 120, for example from computing device 160 via user input 166. In instance where processors 120 provide templates for entering inputs of FIG. 3, these templates may further allow inputs on animations to be entered, or separate templates may be generated for entering inputs on animations. As described above with reference to FIG. 2, the templates may be viewed on display 165 and downloaded to computing device 160, and modified with inputs on animations via user input 166. The modified templates may be uploaded to the web-based application, or an address (e.g. URL) of the modified template may be provided to the web-based application. Processors 120 may then access the inputs on the animations in the modified template.

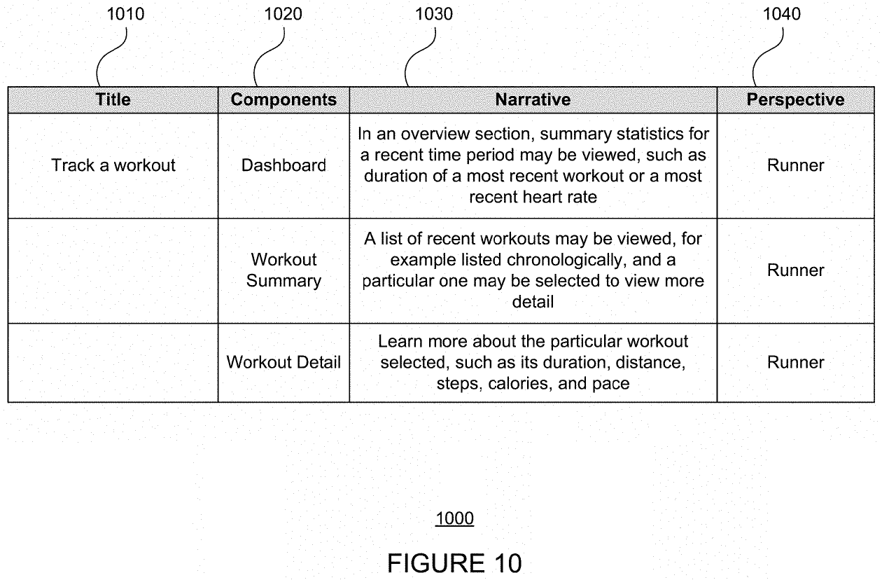

The additional inputs on the animations may be received by processors 120 in a simple form, such as in a tabular form or some other textual form. FIG. 10 illustrates example additional inputs on animations in a tabular form. The inputs of FIG. 10 and the inputs of FIG. 3 may be received as separate sheets of a spreadsheet. In some instances, instead of tabular inputs, the sequence of components for walkthrough may be selected directly on an interactive display of the visualization shown in FIG. 4.

The example of FIG. 10 is for the exercise tracking application of FIGS. 3-9, where table 1000 includes inputs associated with a sequence of two or more components of the exercise tracking application. Thus, such a sequence corresponds to a sequence of displays or screens that a user may view while interacting with the exercise application on a GUI. Although a particular example is shown, the sequence may generally correspond to a plurality of components in an organizational chart, technical architecture, process diagram, or any hierarchical structure. For example, the sequence may be a sequence of webpages that a user may see while interacting with a website, a sequence of storages that a piece of data may be transmitted and/or saved in a cloud computing system, a sequence of parts that together perform an operation in an electronic device, a sequence of processes that generate a result for a software program, a sequence of topics related to one character in a book, a sequence of departments for developing a product in a corporation, a sequence of physical locations visited on one day of a tour, etc.

As shown in table 1000, inputs on the animation may include a title 1010 for the animated walkthrough. The title 1010 “Track a workout” thus summarizes that the sequence of components represent how a workout may be tracked in the exercise tracking application. Table 1000 further includes components 1020. In this regard, the components to be used in the animated walkthrough may be listed in the order they should appear. Thus as shown, the sequence starts with component “Dashboard,” followed by component “Workout Summary,” followed by component “Workout Detail.” Although in this example, the components 1020 are listed by their identifiers 310, in other examples the components may be listed in other ways, such as by their addresses 630.

The table 1000 may include additional information. For instance, table 1000 may further include narrative 1030 accompanying the animated walkthrough. In this example, the narrative 1030 explains what each component in the walkthrough shows in terms of tracking a workout. Table 1000 may also include perspective 1040 of the animated walkthrough. In this example, the perspective 1040 of the animated walkthrough is a runner. Other alternative or additional information may be provided for the walkthrough.

Further, multiple animated walkthroughs may be generated using inputs as shown in FIG. 10, for example, in the same table 1000 under a different Title, or in a separate table. For example, inputs for each of a plurality of animated walkthroughs may be provided in separate sheets of a spreadsheet.

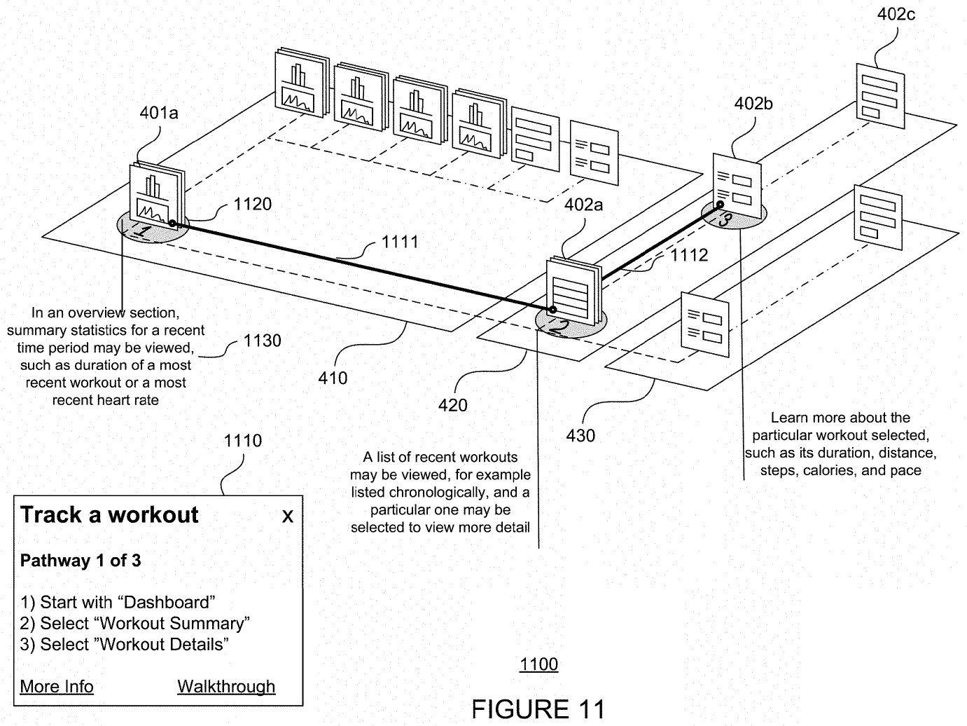

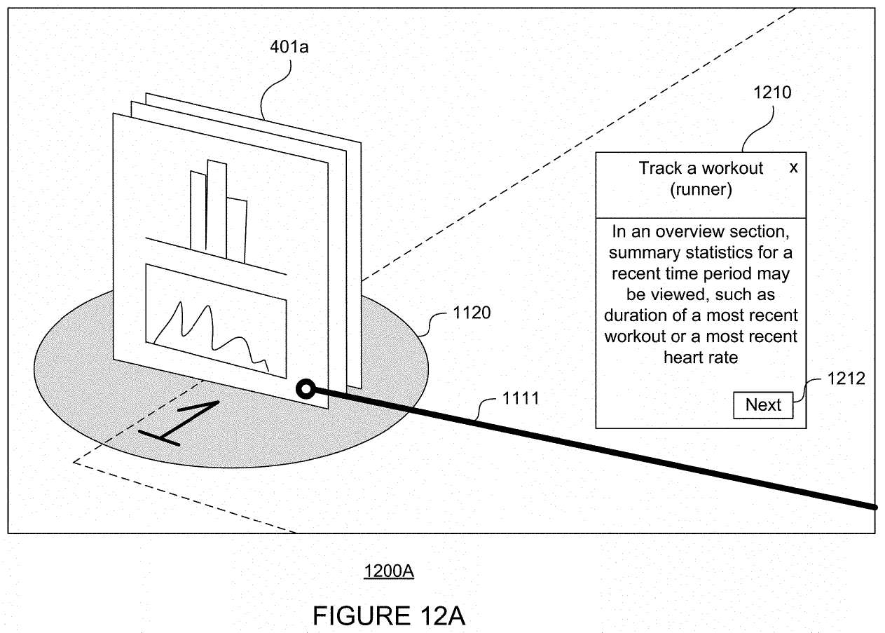

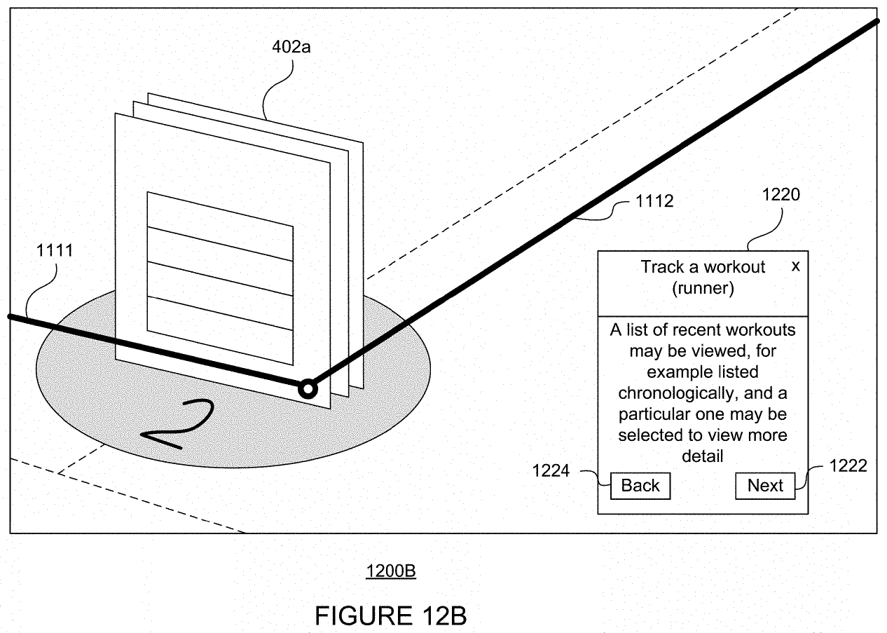

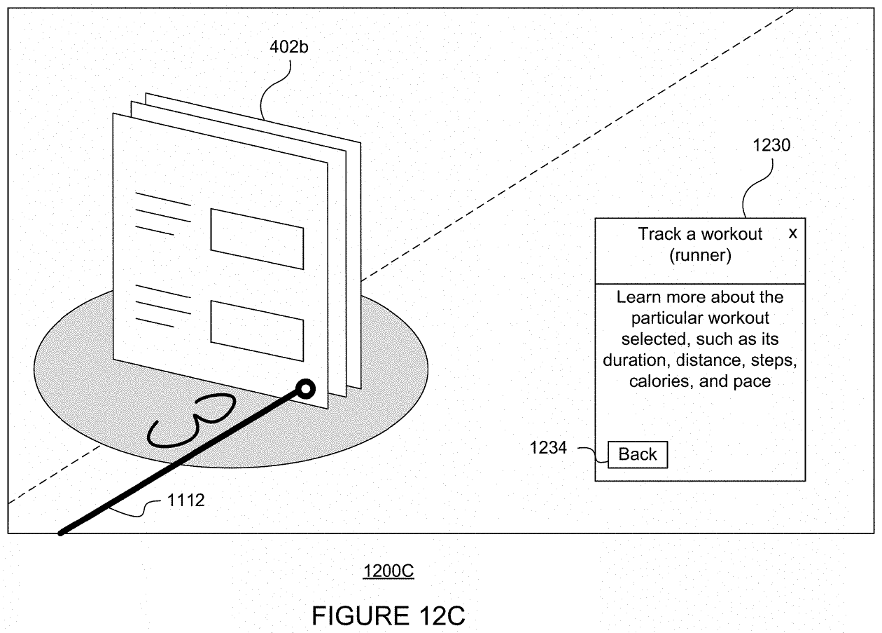

Based on the additional inputs, one or more animated walkthroughs may be generated. FIG. 11 shows an example overview summarizing an example animated walkthrough generated based on inputs of FIG. 10. FIGS. 12A-12C shows example sequence of views in example animated walkthrough generated using the inputs of FIG. 10. In some instances, animated walkthroughs may be generated as separate visualizations or images. In other instances, animated walkthroughs may be generated as an overlay on the visualization showing the plurality of components. For example, the animated walkthrough 1200A-1200C may be generated as overlay on the visualization 400 of FIG. 4, the visualization 700 of FIG. 7, or the visualization 800 of FIG. 8.

Referring to FIG. 11, a walkthrough overview 1100, is shown as an overlay on the visualization of FIG. 7, although some connections from FIG. 8 are also shown. The walkthrough overview includes a summary 1110, which provides information about the walkthrough, such as its title “Track a workout,” and lists the components in the walkthrough “Dashboard,” “Workout Summary,” and “Workout Details.” The summary 1110 further provides that the walkthrough is one of a plurality of walkthroughs possible, as indicated by “Pathway 1 of 3.” For example, another walkthrough for tracking the workout may start from the “Profile” component, and yet another may end at the “Edit Activity” component. The summary 1110 also provides links, such as a “Walkthrough” link that can be selected in order to start the animated walkthrough. The summary 1110 may also include other links, such as a “More Info” link, which for example may lead to textual descriptions or to other walkthroughs. The summary 1110 may include other alterative or additional information, such as statistics, graphics, etc. Although the summary 1110 is provided as a window, in other examples the summary 1110 may be provided in any other format.

The walkthrough overview 1100 further shows the sequence of components involved. For instance, a first line 1111 connects the first component in the sequence, “Dashboard,” to the second component in the sequence, “Workout Summary.” A second line 1112 connects the second component in the sequence “Workout Summary” to the third component in the sequence, “Workout Details.” The sequence of the components are further emphasized, for example by highlights 1120, which may optionally include the order of the component in the sequence. Further as shown, the walkthrough overview 1100 may include narratives 1130 for each step of the walkthrough. As such, the walkthrough overview 1100 provides a high-level view and summary of the animation.

Turning to FIGS. 12A-12C, individual snapshots from the animated walkthrough are illustrated. For example, the view of FIG. 12A may be opened by selecting the “Walkthrough” link in the walkthrough overview 1100. In the first snapshot 1200A of the animated walkthrough, graphic element 401a corresponding to “Dashboard,” which is the first component in the walkthrough, is shown. This first snapshot 1200A further highlights the graphic element 401a and indicates its order in the sequence. Additionally, line 1111 connecting to the next component in the walkthrough is shown.

A window 1210 is also shown in this first snapshot 1200A, which includes information for the first component in the sequence. For example, the window 1210 includes title of the walkthrough, that the walkthrough is from the perspective of a runner, and a narrative describing what the runner may do using the “Dashboard” component. The window 1210 may include links, such as a “Next” link 1212, which may be selected to move to a next view of the animated walkthrough. Additional links may be provided, such as “End” to stop the animation, or “Start from Beginning,” etc. In other examples, the animated walkthrough may be timed, such that the animation automatically moves to the next view in the walkthrough after a predetermined time.

FIG. 12B shows the second snapshot 1200B in the animated walkthrough. In this second snapshot 1200B, graphic element 402a corresponding to “Workout Summary,” which is the second component in the walkthrough, is shown, and highlighted with its order in the sequence. Additionally, this second snapshot 1200B shows both the line 1111 connecting to the previous component of the walkthrough, and line 1112 connecting to the next component in the walkthrough. A window 1220 is also shown in this second snapshot 1200B, which includes information for the second component in the sequence such as the title of the walkthrough, that the walkthrough is from the perspective of a runner, and a narrative describing what the runner may do using the “Workout Summary” component. The window 1220 may include links, such as a “Next” link 1222, which may be selected to move to a next view of the animated walkthrough, and a “Back” link 1224, which may be selected to go back to the previous view of the animated walkthrough.

FIG. 12C shows the third snapshot 1200C in the animated walkthrough. In this third snapshot 1200C, graphic element 402b corresponding to “Workout Details,” which is the third component in the walkthrough, is shown, and highlighted with its order in the sequence. Additionally, this third snapshot 1200C shows the line 1112 connecting to the previous component in the walkthrough. A window 1230 is also shown in this third snapshot 1200C, which includes information for the third component in the sequence such as the title of the walkthrough, that the walkthrough is from the perspective of a runner, and a narrative describing what the runner may do using the “Workout Detail” component. The window 1230 may include links, such as a “Back” link 1234, which may be selected to go back to the previous view of the animated walkthrough.

FIGS. 12A-12C illustrates an example walkthrough, in other examples, additional and/or alternative information may be presented, and may be presented in other formats. For example, crosslinked components may be listed in each step of the sequence, showing other options that may be explored from a current component. As another example, although information on each step of the walkthrough in this example is provided in windows, in other examples the text may be directly on the visualization. In some instances, the display may be interactive, such as allowing for zooming in and/or out, changing of perspective, etc.

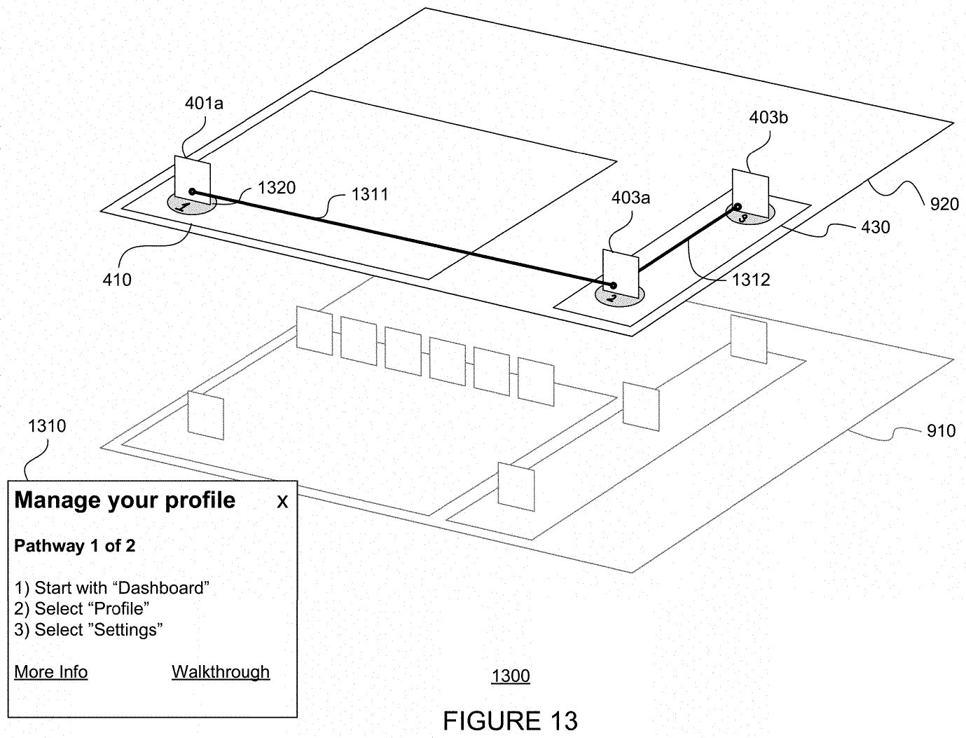

FIG. 13 shows another example overview summarizing another example animated walkthrough. Individual snapshots of this example walkthrough are not presented, FIG. 13 is presented to illustrate an example with multiple elevation levels. Referring to FIG. 13, a walkthrough overview 1300 is shown as an overlay on the visualization of FIG. 9. The walkthrough overview 1300 includes many similar features as the walkthrough overview 1100 of FIG. 1, such as a summary 1310, a line 1311 connecting between the first and second components in the walkthrough, a line 1312 connecting the second and third components in the walkthrough, and highlight 1320 indicating order in the walkthrough. However, in this walkthrough overview 1300, components of the walkthrough are further highlighted by being presented at a different elevation level 920 as the other components of the exercise tracking application (shown as faded). Where a visualization includes many components, separating the components of a walkthrough in a different elevation level may provide a clearer illustration. Further in this regard, where multiple walkthroughs are generated, they may be presented in the walkthrough overview in different elevation levels, which may be a helpful visual aid to show complex and/or numerous pathways.

The technology is advantageous because it provides visualization of complex architecture in 2.5D or 3D views. Features of the technology allows users to provide simple inputs, based on which complex illustrative visualizations may be generated quickly, instead of requiring significant design skills and/or effort. The technology further allows users to generate animations of user experience and/or flow of information using simple inputs, also without requiring significant design skills and/or effort.

Unless otherwise stated, the foregoing alternative examples are not mutually exclusive, but may be implemented in various combinations to achieve unique advantages. As these and other variations and combinations of the features discussed above can be utilized without departing from the subject matter defined by the claims, the foregoing description of the embodiments should be taken by way of illustration rather than by way of limitation of the subject matter defined by the claims. In addition, the provision of the examples described herein, as well as clauses phrased as “such as,” “including” and the like, should not be interpreted as limiting the subject matter of the claims to the specific examples; rather, the examples are intended to illustrate only one of many possible embodiments. Further, the same reference numbers in different drawings can identify the same or similar elements.

1. A method, comprising:
receiving, by one or more processors, inputs associated with a plurality of components of an organizational structure, wherein the inputs include an identifier, a first classification, and a second classification for each of the plurality of components;
determining, by the one or more processors based on the inputs, relationships between the plurality of components within the organizational structure; and
generating, by the one or more processors, a visualization with a plurality of graphic elements corresponding to the plurality of components, wherein the plurality of graphic elements are arranged in a plurality of sections based on the first classifications, and arranged in one or more hierarchies based on the second classifications, and wherein the visualization includes one or more indications of the relationships between the plurality of components within the organizational structure.

2. The method of claim 1, further comprising:
receiving, by the one or more processors, one or more additional classifications for each of the plurality of components;
generating, by the one or more processors, icons as the plurality of graphic elements, wherein appearance of the icons are based on the one or more additional classifications.

3. The method of claim 1, further comprising:
receiving, by the one or more processors, additional input indicating that two or more of the plurality of components are connected;
generating, by the one or more processors based on the additional input, one or more connections for the corresponding graphic elements connecting to adjacent graphic elements in the visualization.

4. The method of claim 1, further comprising:
receiving, by the one or more processors, additional input indicating that two or more of the plurality of components are crosslinked;
generating, by the one or more processors based on the additional input, one or more crosslinks for the corresponding graphic elements to non-adjacent graphic elements in the visualization.

5. The method of claim 4, wherein the one or more crosslinks are hidden and appear in the visualization only when one or more of the crosslinked graphic elements is selected.

6. The method of claim 1, further comprising:
receiving, by the one or more processors, one or more external links associated with one or more of the plurality of components, wherein the corresponding graphic elements are generated with the one or more external links.

7. The method of claim 1, further comprising:
receiving, by the one or more processors, an address for each of the plurality of graphic elements, wherein the plurality of graphic elements are further arranged on the visualization based on the addresses.

8. The method of claim 1, wherein the visualization shows the plurality of graphic elements as two-and-a-half dimensional or three dimensional objects in a perspective view.

9. The method of claim 8, further comprising:
receiving, by the one or more processors, a command to change a view of the visualization;
generating, by the one or more processors based on the command, a two dimensional view of the visualization.

10. The method of claim 1, wherein the inputs are received in a tabular form.

11. The method of claim 1, further comprising:
providing, by the one or more processors, a web-based application with templates for entering inputs, wherein the inputs associated with the plurality of components are received through the web-based application.

12. The method of claim 1, further comprising:
receiving, by the one or more processors, an elevation for each of the plurality of components, wherein the plurality of graphic elements are further arranged on the visualization in a layered stack based on the elevations.

13. The method of claim 1, further comprising:
receiving, by the one or more processors, additional inputs specifying one or more sequences including two or more of the plurality of components;
generating, by the one or more processors based on the additional inputs, one or more animated walkthroughs of graphic elements corresponding to the one or more sequences.

14. The method of claim 13, wherein the one or more animated walkthroughs are generated as an overlay on the visualization.

15. The method of claim 13, wherein the inputs and the additional inputs are received in a tabular form as separate sheets.

16. A system, comprising:
one or more processors, configured to:
receive inputs associated with a plurality of components of an organizational structure, wherein the inputs include an identifier, a first classification, and a second classification for each of the plurality of components;
determine, based on the inputs, relationships between the plurality of components within the organizational structure; and
generate a visualization with a plurality of graphic elements corresponding to the plurality of components, wherein the plurality of graphic elements are arranged based on the relationships between the plurality of components, and wherein the visualization includes one or more indications of the relationships between the plurality of components within the organizational structure.

17. The system of claim 16, wherein the one or more processors are further configured to:
receive one or more additional classifications for each of the plurality of components;
generate icons as the plurality of graphic elements, wherein appearance of the icons are based on the one or more additional classifications.

18. The system of claim 16, wherein the one or more processors are further configured to:
receive an elevation for each of the plurality of components, wherein the plurality of graphic elements are further arranged on the visualization in a layered stack based on the elevations.

19. The system of claim 16, wherein the one or more processors are further configured to:
receive additional inputs specifying one or more sequences including two or more of the plurality of components;
generate, based on the additional inputs, one or more animated walkthroughs of graphic elements corresponding to the one or more sequences.

20. A computer-readable storage medium storing instructions executable by one or more processors for performing a method, comprising:
receiving inputs associated with a plurality of components of an organizational structure, wherein the inputs include an identifier, a first classification, and a second classification for each of the plurality of components;
determining, based on the inputs, relationships between the plurality of components within the organizational structure; and
generating a visualization with a plurality of graphic elements corresponding to the plurality of components, wherein the plurality of graphic elements are arranged in a plurality of sections based on the first classifications, and arranged in one or more hierarchies based on the second classifications, wherein the visualization includes one or more indications of the relationships between the plurality of components within the organizational structure.
